SEQUENCE LISTING (24 sequences)

SEQ ID NO: 1 - DNA, 48 bp, Artificial
  primer PTSG1
tgcccgccgttgtatcgcatgttatggcagggggatcgatcctctaga

SEQ ID NO: 2 - DNA, 45 bp, Artificial
  primer PTSG2
tgcagcaaccagagccggtgccatttcgctgggccgacaggcttt

SEQ ID NO: 3 - DNA, 48 bp, Artificial
  primer PTSG3
tgggcgtcggttccgcgaatttcagctggctgcccgccgttgtatcgc

SEQ ID NO: 4 - DNA, 48 bp, Artificial
  primer PTSG4
gaggttagtaatgttttctttaccaccaaatgcagcaaccagagccgg

SEQ ID NO: 5 - DNA, 48 bp, Artificial
  primer PYKF1
tggacgctggcatgaacgttatgcgtctgagggtagatttcagtgcaa

SEQ ID NO: 6 - DNA, 48 bp, Artificial
  primer PYKF2
cgcctttgctcagtaccaactgatgagccggggttccattcaggtcga

SEQ ID NO: 7 - DNA, 48 bp, Artificial
  primer PYKF3
ccgaatctgaagagatgttagctaaaatgctggacgctggcatgaacg

SEQ ID NO: 8 - DNA, 48 bp, Artificial
  primer PYKF4
aagtgatctctttaacaagctgcggcacaacgcctttgctcagtacca

SEQ ID NO: 9 - DNA, 48 bp, Artificial
  primer MQ01
ggcataccatgccggatgtggcgtatcattggggtttaagggcaccaa

SEQ ID NO: 10 - DNA, 48 bp, Artificial
  primer MQ02
gaactacggcgagatcacccgccagttaatgccccgggctttgcgccg

SEQ ID NO: 11 - DNA, 48 bp, Artificial
  primer MQ03
tggcgcgtcttatcagcatacgccacatccggcataccatgccggatg

SEQ ID NO: 12 - DNA, 48 bp, Artificial
  primer MQ04
agccacgcgtacggaaattggtaccgatgtgaactacggcgagatcac

SEQ ID NO: 13 - DNA, 48 bp, Artificial
  primer SDH1
cagtcagagaatttgatgcagttgtgattgatcgggggggggggaaag

SEQ ID NO: 14 - DNA, 48 bp, Artificial
  primer SDH2
atcggctctttcaccggatcgacgtgagcgatcccaattctgattaga

SEQ ID NO: 15 - DNA, 48 bp, Artificial
  primer SDH3
gttgtggtgtggggtgtgtgatgaaattgccagtcagagaatttgatg

SEQ ID NO: 16 - DNA, 48 bp, Artificial
  primer SDH4
atcatgtagtgacaggttgggataaccggaatcggctctttcaccgga

SEQ ID NO: 17 - DNA, 48 bp, Artificial
  primer ACEBA1
gcaccttgtgatggtgaacgcaccgaagaacgagctcggtacccgggc

SEQ ID NO: 18 - DNA, 48 bp, Artificial
  primer ACEBA2
ctttcgcctgttgcagcgcctgaccgccagcaatagaccagttgcaat

SEQ ID NO: 19 - DNA, 48 bp, Artificial
  primer ACEBA3
gacgcgccgattactgccgatcagctgctggcaccttgtgatggtgaa

SEQ ID NO: 20 - DNA, 48 bp, Artificial
  primer ACEBA4
atcccgacagatagactgcttcaatacccgctttcgcctgttgcagcg

SEQ ID NO: 21 - DNA, 49 bp, Artificial
  primer PYKA1
cacctggttgtttcagtcaacggagtattacatcgggggggggggaaag

SEQ ID NO: 22 - DNA, 47 bp, Artificial
  primer PYKA2
gtggcgttttcgccgcatccggcaacgtacatcccaattctgattag

SEQ ID NO: 23 - DNA, 48 bp, Artificial
  primer PYKA3
ttatttcattcggatttcatgttcaagcaacacctggttgtttcagtc

SEQ ID NO: 24 - DNA, 49 bp, Artificial
  primer PYKA4
gttgaactatcattgaactgtaggccggatgtggcgttttcgccgcatc

(19) United States
(12) Patent Application Publication
(10) Pub. No.: US 2009/0215048 A1
(43) Pub. Date: Aug. 27, 2009

(54) METHOD OF IN-SILICO IMPROVEMENT OF ORGANISMS USING THE FLUX SUM OF METABOLITES

(75) Inventors: Sang Yup Lee, Daejeon (KR); Tae Yong Kim, Gyeonggi-do (KR); Dong Yup Lee, Gyeonggi-do (KR)

Correspondence Address: INTELLECTUAL PROPERTY / TECHNOLOGY LAW, PO BOX 14329, RESEARCH TRIANGLE PARK, NC, 27709, US

(73) Assignee: KOREA ADVANCED INSTITUTE OF SCIENCE AND TECHNOLOGY, Daejeon (KR)

(21) Appl. No.: 11/994,330

(22) PCT Filed: Oct. 14, 2005
(86) PCT No.: PCT/KR05/03442

§ 371 Date: Dec. 29, 2007

(30) Foreign Application Priority Data
Jul. 11, 2005 (KR) 10-2005-0062404

(51) Int. Cl. C12Q 1/68; G06F 19/00; C12N 15/00; C12N 15/74; C12P 7/46

(52) U.S. Cl. 435/6; 702/19; 435/440; 435/471; 435/145

(57) ABSTRACT

The present invention relates to an in silico method for improving an organism on the basis of the flux sum (φ) of metabolites, and more particularly to a method for screening key metabolites that increase the production yield of a useful substance, the method comprising defining the metabolite utilization of an organism for producing a useful substance as flux sum and perturbing the flux sum, as well as a method for improving an organism producing a useful substance, the method comprising deleting and/or amplifying genes associated with the aforementioned screened key metabolites. According to the present invention, the correlation between specific metabolites and useful substance production can be exactly predicted, so that it is possible to develop an organism having increased useful substance production by introducing and/or amplifying and/or deleting genes expressing enzymes associated with the specific metabolites. In addition, it is also possible to increase the production of a useful substance by adding specific metabolites during culture.

### TECHNICAL FIELD

The present invention relates to an in silico method for improving an organism on the basis of the flux sum (φ) of metabolites, and more particularly to a method for screening key metabolites that increase production yield of a useful substance, the method comprising defining the metabolite utilization of an organism for producing a useful substance as flux sum, perturbing the flux sum, as well as a method for improving an organism producing a useful substance, the method comprising deleting and/or amplifying genes associated with the aforementioned screened key metabolites.

### BACKGROUND ART

Biological methods for producing useful substances using microorganisms have advantages in that they are more eco-friendly and provide final products having high stability, compared to conventional chemical methods. However, most of these biological methods have low production yield and produce many byproducts in addition to the desired substance, and thus have disadvantages regarding the isolation and purification of the product. For this reason, these biological methods have encountered limitations on their industrial use and there have been many attempts to overcome these limitations, but attempts up to now have been mainly focused on the development of an efficient production process or isolation process.

Since these methods are not based on the manipulation of metabolic property itself of production strains, it is difficult to expect a greater degree of result. Particularly, since the process and isolation technologies have recently reached a stabilized stage, it is difficult to expect productivity to be greatly increased by the improvement of these technologies. Accordingly, operations to fundamentally improve the productivity and properties of strains by manipulating the metabolic network of microorganisms are more desirable.

Strain improvement with metabolic engineering is currently performed by methods based on overexpressing one or two enzymes, or introducing or removing simple metabolic circuits, but in many cases, they do not provide good results as expected. In addition, for the production of substances requiring changes in the complex metabolic fluxes, strains improved by metabolic engineering can be hardly used. This is because complex metabolic circuits are not sufficiently understood for directed engineering. Recombinant gene technology for the manipulation and introduction of a metabolic network is much more advanced, whereas analysis and prediction technology based on a metabolic network has only recently become feasible with rapidly increasing genomic information. Particularly, with the development of the mathematical representation of the organisms' metabolism and its simulation using optimization techniques, it is becoming possible to predict metabolic pathway reactions occurring after deletion or addition of specific genes on a computer (Lee et al., Trends Biotechnol., 23:349, 2005).

Mathematical models for analyzing cell metabolism can be generally divided into two categories, i.e., dynamic and static models. The dynamic models simulate the dynamic state of cells by predicting intracellular changes with respect to time. However, dynamic models require many physiological parameters, and thus is very time-consuming and difficult in terms of mathematical complexity. On the other hand, in the case of models containing the information of regulatory mechanisms and in the case of static mathematical models that only consider stoichiometry of biochemical reactions, only the mass balance of biochemical reactions and the information of cell composition are used to determine an ideal metabolic flux space that cells can attain. This metabolic flux analysis (MFA) technique shows the ideal metabolic flux of cells and allows exact simulation and prediction of the cell behaviors, even though it does not require physiological parameters (Papin, J. et al., Nature Reviews Molecular Cell Biology, 6:99, 2005).

The metabolic flux analysis can be generally used to calculate, for example, the maximum production yield of a desired metabolite by strain improvement, and the calculated values can be used to understand metabolic pathway properties inside strains. Particularly, there have been various studies that apply the metabolic flux analysis method to predict, for example, changes in metabolic pathway fluxes, caused by deletion or addition of genes. Such studies have been conducted mainly in connection with deletion of specific target genes for increasing the production of a useful substance. Furthermore, there have been efforts to find an ideal combination of genes, which simultaneously satisfies two purposes, i.e., an increase in the production of a useful substance and the growth of an organism (Pharkya et al., Biotechnol. Bioeng., 84:887, 2003; US 2004/0009466A1).

However, in using the existing mathematical models to simulate reactions caused by deletion and addition of a plurality of genes, there is a problem in that gene combinations increase at an exponential rate. Particularly in the case of simulations where at least three genes are deleted, combinations of genes responsible for 1,000 metabolic reactions will be more than billion combinations. To perform such simulations, much time is required even when a computer having the best performance is used. When combinations of 4 or 5 genes are considered, situation becomes even worse. As a result, the prediction of effects on the deletion and addition of genes, conducted before experiments, becomes meaningless.

Meanwhile, the existing metabolic flux analysis methods and the simulations resulting from deletion and addition of genes have a shortcoming in that they cannot examine the properties of internal metabolites, since the concentration of metabolites is assumed not to be changed under the assumption of a quasi-steady state. If internal metabolic pathways are actually altered by genetic manipulation, how to change the pool of specific internal metabolites will become important in many cases. Accordingly, if a simulation is conducted by metabolic flux analysis for the production of useful substances by microorganisms, a more effective methodology will be required.

### DISCLOSURE OF THE INVENTION

Accordingly, the present inventors have made extensive efforts to find a method capable of effectively increasing the production of a useful target substance and, as a result, found that specific key metabolites involved in the production of the useful substance can be screened by mathematically defining and then using the flux sum to quantitatively analyze the metabolite utilization of a host organism for producing a useful substance, thereby completing the present invention.

It is therefore an object of the present invention to provide a method for screening specific key metabolites involved in the production of a useful substance.

Another object of the present invention is to provide a method for improving an organism producing a useful substance, the method comprising deleting and/or amplifying genes associated with the screened specific metabolites.

To achieve the above objects, the present invention provides a method for screening key metabolites responsible for the increased production of a useful substance. The method consists of:
(a) selecting a host organism (except for human beings) for producing the useful target substance, and constructing the metabolic network model of the selected organism;
(b) defining the utilization of each of metabolites of the constructed metabolic network as flux sum (φ) represented by equation 1 below, and determining the value φ of the metabolites; and
(c) perturbing the values of φ of the metabolites so as to screen key metabolites involved in increasing the production yield of the useful substance:

$$\begin{matrix}{Φ}_{i} = \vert {f}_{i n} \vert \\ = \vert {f}_{out} \vert \\ = 1 / 2 {\sum }_{j} \vert {S}_{ij} {v}_{j} \vert\end{matrix} \qquad [ Equation 1 ]$$

wherein φᵢ represents the utilization of an i^{th} metabolite, fᵢₙ represents the metabolic flux of a reaction where a relevant metabolite is consumed with respect to the i^{th} metabolite, fₒᵤₜ represents the metabolic flux of a reaction where the useful target substance is produced with respect to the i^{th} metabolite, Sᵢⱼ represents the stoichiometric coefficient of the i^{th} metabolite in the j^{th} reaction, and vⱼ represents the metabolic flux vector of the j^{th} pathway.

In the screening method, the perturbation in the step (c) is performed by increasing and/or attenuating the value φ. More specifically, if an attenuation and/or increase in the value φ of a specific metabolite leads to an increase in the yield of the useful target product, the specific metabolite can be clustered and screened.

In one embodiment, the present invention provides a method for improving an organism producing a useful substance. The method consists of the following steps: (a) selecting a host organism for producing a useful target substance, and constructing the metabolic network model of the selected organism; (b) defining the utilization of each metabolite of the constructed metabolic network as flux sum (φ) represented by Equation 1, and determining the value φ of the metabolites; (c) clustering and screening a specific metabolite, if an attenuation in the value φ of the specific metabolite leads to an increase in the yield of the useful target product; (d) selecting genes to be deleted from a metabolic network associated with the specific metabolite screened in the step (c); and (e) deleting the genes selected in the step (d) from the host organism so as to construct a mutant of the host organism.

In the above improvement method, the useful target substance is preferably succinic acid, and the host organism is preferably a microorganism producing succinic acid. Also, the genes to be deleted for the production of succinic acid are selected from a group consisting of ptsG, pflb, pykF and pykA.

In another embodiment, the present invention provides a method for improving an organism producing a useful substance. The method consists of the following steps: (a) selecting a host organism for producing a useful target substance, and constructing the metabolic network model of the selected organism; (b) defining the utilization of each metabolite of the constructed metabolic network as flux sum (φ) represented by Equation 1 and determining the value φ of the metabolites; (c) clustering and screening a specific metabolite, if an increase in the value φ of the specific metabolite leads to an increase in the yield of the useful target product; (d) selecting genes to be amplified from a metabolic network associated with the specific metabolite screened in the step (c); and (e) introducing the genes selected in the step (d) into the host organism for gene amplification, so as to construct a mutant of the host organism.

In still another embodiment, the present invention provides a method for improving an organism producing a useful substance. The method consists of the following steps: (a) selecting a host organism for producing a useful target substance, and constructing the metabolic network model of the selected organism; (b) defining the utilization of each metabolite of the constructed metabolic network as flux sum (φ) represented by Equation 1 and determining the value φ of the metabolites; (c) clustering and screening the metabolites, if an attenuation or increase in the value φ of the metabolites leads to an increase in the yield of the useful target product; (d) selecting genes to be deleted and/or amplified from a metabolic network associated with the metabolites screened in the step (c); and (e) deleting the genes to be deleted, which are selected in the step (d) from the host organism while introducing the genes to be amplified into the host organism and/or amplifying the genes in the host organism, so as to construct a mutant of the host organism.

In the inventive method for improving the organism producing the useful substance may additionally comprise the step (f) of culturing the mutant constructed in the step (e) so as to experimentally verify the production of the useful substance.

In another aspect, the present invention provides a method for producing a useful substance which is culturing the organism improved by the aforementioned method.

In one embodiment, the present invention provides a method for producing a useful substance by the culture of an organism, which comprises supplying a metabolite in the culture process. The procedure comprising the steps of: (a) selecting a host organism (except for human beings) for producing a useful target substance, and constructing its metabolic network model; (b) defining the utilization of each metabolite of the constructed metabolic network as flux sum (φ) represented by Equation 1, and determining the value φ of the metabolites; and (c) clustering and screening a specific metabolite, if an increase in the value φ of the specific metabolite leads to an increase in the yield of the useful target product.

In Equation 1 in the present invention, fᵢₙ and fₒᵤₜ are preferably represented by Equations 2 and 3 below, respectively:

$$\begin{matrix}{f}_{i n} = {\sum }_{j}^{ingoing} \vert {S}_{ij} {v}_{j} \vert & [ Equation 2 ] \\ {f}_{out} = {{\sum }^{outgoing}}_{j} \vert {S}_{ij} {v}_{j} \vert & [ Equation 3 ]\end{matrix}$$

wherein Sᵢⱼ represents the stoichiometric coefficient of the i^{th} metabolite in the j^{th} reaction, and vⱼ represents the metabolic flux vector of the j^{th} pathway.

In the present invention, the host organism is preferably a microorganism. Also, the useful target substance is succinic acid, and the host organism is a microorganism capable of producing succinic acid.

As used herein, the term “perturbation” refers to a manipulation perturbing a group of all metabolites by the application of a specific external factor so as to find a metabolite having the desired property.

As used herein, the term “clustering” is intended to include a method and process of grouping metabolites showing similar patterns from a group of metabolites resulting from perturbation of all metabolites.

As used herein, the “deletion” of genes encompasses all operations of rendering specific genes inoperative in an organism, such as removing or altering all or part of the base sequences of the genes, and the “amplification” of genes encompasses all operations of increasing the expression levels of the relevant genes by manipulating all or part of the base sequences of the genes to be replicated in an organism in large amounts.

As used herein, the term “culture” is defined to encompass not only the culture of microorganisms, such as bacteria, yeasts, fungi, and animal and plant cells, but also the cultivation of plants and the breeding of animals.

Other features and embodiments of the present invention will be more clearly understood from the following detailed description and the appended claims.

### BRIEF DESCRIPTION OF DRAWINGS

FIG. 1 is a block diagram showing the inventive method for increasing the production of a useful substance by the analysis of key metabolites using flux sum.

FIG. 2 shows an example of metabolic fluxes of a reaction where a relevant metabolite is consumed with respect to i^{th} metabolite. In FIG. 2, fᵢₙ consists of three metabolic fluxes, and fₒᵤₜ consists of two metabolic fluxes.

FIG. 3 shows a process of screening a specific metabolite, if an attenuation in the flux sum (φ) of the specific metabolite leads to an increase in the metabolic flux of a useful target substance.

FIG. 4(A) shows the profile of biomass formation rate (A) and the profile of bioproduct formation rate (B) according to the flux sum of each metabolite.

FIG. 5 shows the profile of biomass formation rate by the perturbation of flux sum of each metabolite.

FIG. 6 shows the formation rate of a useful product (succinic acid) by the perturbation of flux sum of each metabolite.

FIG. 7 shows biomass formation rate and succinic acid production rate (y-axis) by the perturbation of flux sum of pyruvate (x-axis).

FIG. 8 shows metabolic reactions and the values of metabolic fluxes with respect to pyruvate in E. coli.

FIG. 9 shows a metabolic network with respect to pyruvate in E. coli.

### DETAILED DESCRIPTION OF THE INVENTION, AND PREFERRED EMBODIMENTS THEREOF

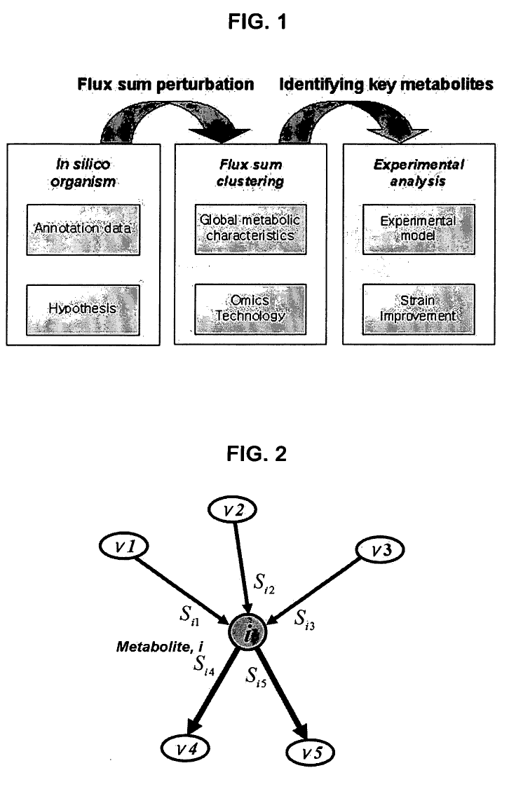

Hereinafter, the present invention will be described in detail. FIG. 1 shows the concept of a method for increasing the production of a useful substance by the analysis of key metabolites using flux sum according to the present invention. Namely, the present invention provides a method for screening key metabolites involved in increasing the production yield of a useful target product by clustering, electing a host organism producing a useful substance, constructing the metabolic network model of the selected organism, determining the flux sum values (φ) of metabolites in the constructed metabolic pathway network, and perturbing the flux sum. The present invention will now be described in detail.

### 1. Metabolic Network Construction

In the present invention, a new metabolic flux analysis system was constructed using an E. coli mutant as a host strain for producing a useful substance. This system comprises most of the metabolic network of E. coli. For E. coli, new metabolic network consists of 979 biochemical reactions, and 814 metabolites are considered to be in the metabolic network. The biological composition of E. coli, which is a stoichiometric demand of each constituent of E. coli in the objective function of metabolic flux analysis to calculate the biomass formation rate of a strain, was constructed as disclosed in the prior literature (Neidhardt et al., Escherichia coli and Salmonella: Cellular and Molecular Biology, 1996).

### 2. Definition and Perturbation of Flux Sum

### (1) Flux Sum

If all the metabolites, their biochemical reactions and a corresponding stoichiometric matrix (Sᵢⱼ^{T}; the stoichiometric coefficient of the i^{th} metabolite in j^{th} reaction with time) are known, a metabolic flux vector (vⱼ, the metabolic flux of j pathway) can be calculated, in which a change in metabolite concentration X with time can be expressed as the sum of the fluxes of all metabolic reactions. A change in X with time can be defined as the following equation under the assumption of a quasi-steady state:

S^{T}v=dX/dt=0

wherein S^{T}v is a change in X with time, X is metabolite concentration, and t is time.

Herein, the utilization of fluxes around metabolites is defined as follows in view of metabolites so as to correspond to metabolic fluxes defined in view of metabolic reactions.

Namely, the metabolic flux of a reaction where a relevant metabolite is consumed with respect to i^{th} metabolite is defined as fᵢₙ, and the metabolic flux of a reaction where a relevant metabolite is produced with respect to i^{th} metabolite is defined as fₒᵤₜ, and these metabolic fluxes are represented by Equations 2 and 3 below, respectively.

$$\begin{matrix}{f}_{i n} = {\sum }_{j}^{ingoing} \vert {S}_{ij} {v}_{j} \vert & [ Equation 2 ] \\ {f}_{out} = {{\sum }^{outgoing}}_{j} \vert {S}_{ij} {v}_{j} \vert & [ Equation 3 ]\end{matrix}$$

wherein Sᵢⱼ is the stoichiometric coefficient of the i^{th} metabolite in the j^{th} reaction, and vⱼ is the metabolic flux vector of j pathway.

FIG. 2 shows an example of the metabolic flux of a reaction where relevant metabolites are consumed with respect to i^{th} metabolite. The metabolic fluxes of the reactions shown in FIG. 2 can be defined as follows:

$$\begin{matrix}{f}_{i n} = {{\sum }_{j}}^{ingoing} {S}_{ij} {v}_{j} \\ = {S}_{i 1} {v}_{1} + {S}_{i 2} {v}_{2} + {S}_{i 3} {v}_{3}\end{matrix}$$

$$\begin{matrix}{f}_{out} = {{\sum }_{j}}^{outgoing} {S}_{ij} {v}_{j} \\ = {S}_{i 4} {v}_{4} + {S}_{i 5} {v}_{5}\end{matrix}$$

fᵢₙ and fₒᵤₜ defined above can be considered as the utilization of fluxes around metabolites, since they have the same absolute value under the assumption of a quasi-steady state. In the present invention, the utilization of fluxes around metabolites is named “flux sum” (φ) and defined as equation 1:

$$\begin{matrix}{Φ}_{i} = \vert {f}_{i n} \vert \\ = \vert {f}_{out} \vert \\ = 1 / 2 {\sum }_{j} \vert {S}_{ij} {v}_{j} \vert\end{matrix} \qquad [ Equation 1 ]$$

wherein φᵢ represents the utilization of i^{th} metabolite, fᵢₙ represents the total metabolic flux of reactions where a relevant metabolite is consumed with respect to the i^{th} metabolite, fₒᵤₜ represents the total metabolic flux of reactions where a useful target product is produced with respect to the i^{th} metabolite, Sᵢⱼ represents the stoichiometric coefficient of the i^{th} metabolite in the j^{th} reaction, vⱼ represents the metabolic flux of j pathway.

Flux sum (φ) is an amount newly defined to express the utilization of metabolites, which have not been employed in the existing metabolic analysis method. The more the utilization of relevant metabolites is, the higher the value φ becomes, and the less the utilization of relevant metabolites is, the lower the value φ becomes.

Generally, the existing metabolic flux analysis is based on the assumption of a quasi-steady state, and a change in the concentration of internal metabolites caused by a change in external environment is very immediate, and thus this change is generally neglected and it is assumed that the concentration of internal metabolites is not changed. Namely, the metabolic flux analysis method has a shortcoming in that the property of each metabolite cannot be examined, since a change in the concentration of internal metabolites caused by a change in external environment is very immediate, and thus this change is neglected, whereby it is assumed that the concentration of internal metabolites is not changed.

In the present invention, flux sum (φ) is defined as the utilization of metabolites so as to provide a quantitative base capable of finding key metabolites for increasing the production of a useful substance.

### (2) Flux Sum Perturbation

Value φ is determined from the above definition, and, based on this, value φ for all metabolites can be determined by perturbing the determined value φ or other variables using the following algorithms. Accordingly, the perturbation can be performed by attenuating and/or increasing the value φ as follows.

(i) Attenuation of flux Sum

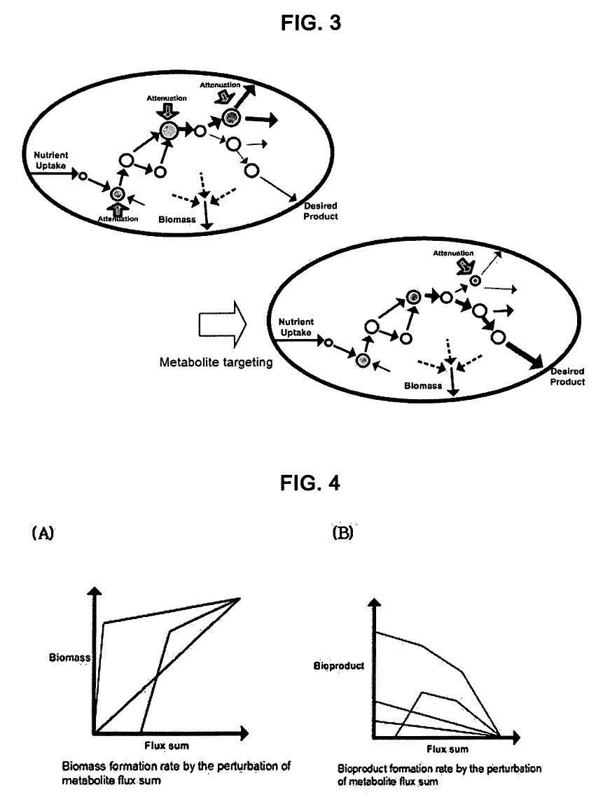

If an attenuation in the value φ of a specific metabolite leads to an increase in the metabolic flux of a relevant useful substance according to the characteristic of the metabolic network, it is possible to screen this specific metabolite (see FIG. 3).

To attenuate the φᵢ of i^{th} internal metabolite to a specific value C, a restriction condition can be set according to the following equation, and biomass formation rate as an objective function can be maximized.

$${Φ}_{i} = 1 / 2 {\sum }_{j} \vert {S}_{ij} {v}_{j} \vert < C$$

If the restriction condition contains absolute value as described above, the DNLP (discontinuous nonlinear programming) problem having discontinous differential value occurs and the optimum value is consequently not found, unlike when solving the general LP (linear programming) problem.

Accordingly, the most reliable method to solve the DNLP problem is to convert the DNLP problem into the LP problem. The method is as follows:
1) Absolute value |Sᵢⱼvⱼ| contained in the φᵢ equation given for the i^{th} metabolite is reformed as follows. First, Sᵢⱼvⱼ=fᵢⱼ−gᵢⱼ is given, wherein fᵢⱼ and gᵢⱼ are any positive variables. Accordingly, if the value |Sᵢⱼvⱼ| is formed into an inequality of Sᵢⱼvⱼ≦fᵢⱼ+gᵢⱼ, the equation is satisfied when fᵢⱼ=0 or gᵢⱼ=0.
2) From the above relationship, the following relationship is obtained.

$${Φ}_{i} = 1 / 2 {\sum }_{j} \vert {S}_{ij} {v}_{j} \vert \leq 1 / 2 {\sum }_{j} ( {f}_{ij} + {g}_{ij} )$$

3) From the above equation, the restriction condition φᵢ>C of the original DNLP form can be modified as follows.

$$1 / 2 {\sum }_{j} ( {f}_{ij} + {g}_{ij} ) \leq C$$

The above method is used as the following algorithm.

Maximization of biomass formation rate:

Sᵢⱼvᵢⱼ=fᵢⱼ−gᵢⱼ, wherein 0≦fᵢⱼ and 0≦gᵢⱼ;

$$1 / 2 {\sum }_{j}^{ } ( {f}_{ij} + {g}_{ij} ) \leq C$$

$${\sum }_{j}^{ } {S}_{kj} {v}_{j} = {b}_{k}$$

(if k^{th} metabolite is an intermediate, bₖ=0);

αⱼ≦vⱼ≦βⱼ.

In the above equations, Sᵢⱼ represents the stoichiometric coefficient of the i^{th} metabolite in the j^{th} reaction, vⱼ represents the metabolic flux vector of j pathway, fᵢⱼ and gᵢⱼ are any positive variables, C is a limit value to which the metabolic flux is to be attenuated, bₖ is the value of metabolic flux toward the outside, αⱼ and βⱼ are limit values that the respective metabolic fluxes can have, and they represent the maximum and minimum values permitted by the respective metabolic fluxes.

The profiles of biomass formation rate and useful substance formation rate by the perturbation of φᵢ of each metabolite can be shown as in FIG. 4. From part (A) of FIG. 4, it can be seen that as the flux sum of each metabolite attenuates, various profiles including the attenuation or maintenance of biomass formation rate are shown. From part (B) of FIG. 4, it can be seen that the release of a useful product of interest to the outside varies depending on the flux sum of which metabolite attenuates. As shown in FIG. 4, since the attenuation in the flux sum of metabolites leads to complex profiles, similar profiles can be grouped by clustering, whereby a group of key metabolites for increasing the yield of a useful target substance can be screened while maintaining biomass formation rate to the greatest possible extent.

### (ii) Increase of Flux Sum

If an increase in the flux sum (φ) of a specific metabolite leads to an increase in the metabolic flux of a relavant useful substance according to the peculiar property of the metabolic network, this specific metabolite can be screened. The screening can be achieved by the following restriction condition, and an actual method and algorithm for this screening are the same as the above method of the attenuation of flux sum:

$$C < {Φ}_{i} = 1 / 2 {\sum }_{j}^{ } \vert {S}_{ij} {v}_{j} \vert$$

(iii) Attenuation and Increase of Flux Sum

If the attenuation and increase of flux sum (φ) of metabolites lead to an increase in the metabolic flux of a useful substance of interest according to the peculiar property of the metabolic network, the metabolites can be screened.

### (3) Experimental Analysis

### (i) Attenuation of Flux Sum

In the case of key metabolites screened by attenuation of flux sum, it is necessary to reduce reactions producing or consuming the relevant metabolites. Accordingly, mutants with deletions of genes associated with reactions producing or consuming the screened metabolites are made, and from the mutants, a strain showing an improvement in the production of a useful substance is selected. The productivity of the selected strain is finally verified through actual culture tests. In Example of the present invention, E. coli mutant strains and recombinant E. coli strains were selected as model systems for applying the aforementioned to the production of succinic acid.

### (ii) Increase of Flux Sum

In the case of key metabolites screened by an increase of flux sum, it is necessary to amplify reactions producing or consuming the relevant metabolites. Accordingly, mutants with amplifications of genes associated with reactions producing or consuming the screened metabolites are made, and from the mutants, a strain showing an improvement in the production of a useful substance is selected. The productivity of the selected strain is finally verified through actual culture tests.

(iii) Increase and Attenuation of Flux Sum

If an increase and attenuation in the value φ of a plurality of metabolites lead to an increase in the metabolic flux of a substance of interest due to the peculiar property of the metabolic network, the metabolites can be screened. Mutants where the screened plurality of metabolites have been amplified and deleted were made and, among the mutants, a strain showing an improvement in the production of a useful substance is selected. The productivity of the selected strain is finally verified through actual culture tests.

### EXAMPLES

Hereinafter, the present invention will be described in more detail by examples. It is to be understood, however, that these examples are for illustrative purpose only and are not construed to limit the scope of the present invention.

Although the following examples particularly illustrate a method for improving a siccinic acid-producing strain using E. coli as a model system, it will be obvious to a person skilled in the art from the disclosure herein that these examples can also be applied to the case of increasing the production of other useful substances in addition to succinic acid, and the case of using model systems of not only microorganisms, such as bacteria, yeasts, fungi, and animal and plant cells, in addition to E. coli, but also animals and plants.

Furthermore, although the following examples illustrate a method for screening key metabolites by attenuating flux sum in order to produce succinic acid as a useful substance, it will be obvious to a person skilled in the art that key metabolites can also be screened by either increasing or attenuating and increasing flux sum according to the present invention.

### Example 1

### Construction of Model System

A new metabolic flux analysis system was constructed using an E. coli mutant strain as a host strain for producing a useful substance. This system comprises most of the E. coli metabolic network. For E. coli, the new metabolic network consists of 979 biochemical reactions, and 814 metabolites are considered in the metabolic network. The biological composition of E. coli for use in an equation of biomass formation rate, which is to be used as an objective function in metabolic flux analysis, was made according to the disclosure of the prior literature (Neidhardt et al., Escherichia coli and Salmonella: Cellular and Molecular Biology, 1996).

### Example 2

### Screening of Key Metabolites by Attenuation of Flux Sum

The value φ for all metabolites was defined as follows, and the value φ for 814 metabolites of E. coli was perturbed in anaerobic condition according to the following algorithms.

Maximization of biomass formation rate:

Sᵢⱼvⱼ=fᵢⱼ−gᵢⱼ, wherein 0≦fᵢⱼ and 0≦gᵢⱼ;

$$1 / 2 {\sum }_{j}^{ } ( {f}_{ij} + {g}_{ij} ) \leq C$$

$${\sum }_{j}^{ } {S}_{kj} {v}_{j} = {b}_{k}$$

(if a k^{th} metabolite is an intermediate, bₖ=0);

αⱼ≦vⱼ≦βⱼ.

In the above equations, Sᵢⱼ represents the stoichiometric coefficient of the i^{th} metabolite in the j^{th} reaction, vⱼ represents the metabolic flux vector of j pathway, fᵢⱼ and gᵢⱼ are any positive variables, C is a limit value to which the metabolic flux is to be attenuated, bₖ is the value of metabolic flux toward the outside, αⱼ and βⱼ are limit values that the respective metabolic fluxes can have, and they represent the maximum and minimum values permitted by the respective metabolic fluxes.

Biomass formation rate (i.e., specific growth rate) was selected as an objective function, and linear programming was used to determine the optimum metabolic flux distribution.

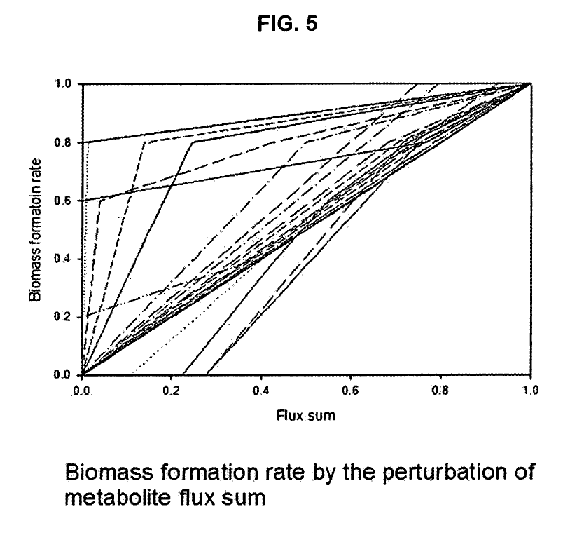

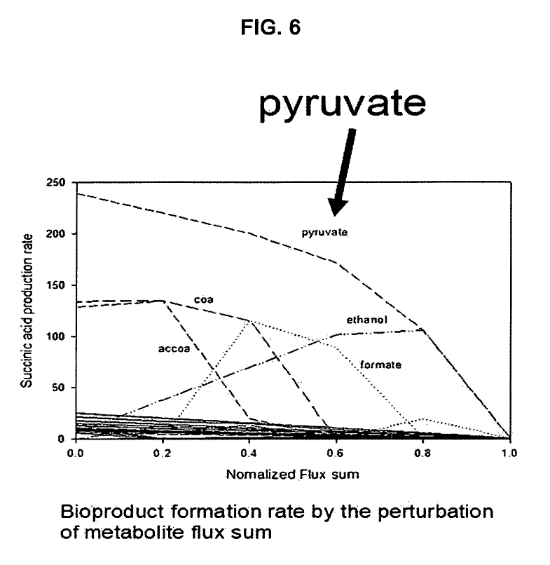

The profiles of biomass formation rate and useful substance formation rate by the perturbation of flux sum of each metabolite are shown in FIGS. 5 and 6. FIG. 5 shows biomass formation rate by the perturbation of flux sum of each metabolite, and FIG. 6 shows the formation rate of a useful product by the perturbation of flux sum of each metabolite.

From FIG. 5, it can be seen that as the flux sum of each metabolite attenuates, various profiles including the attenuation or maintenance of biomass formation rate are shown. From FIG. 6, it can be found that an increase in the metabolic flux of a useful product (succinic acid herein) of interest varies depending on the flux sum of which metabolite attenuates. As such, since the attenuation in the flux sum of metabolites leads to complex profiles, similar profiles can be grouped by clustering, whereby a group of key metabolites for increasing the yield of a useful target substance can be screened while maintaining biomass formation rate to the greatest possible extent. From FIG. 6, it can be seen that, in the case of the production of succinic acid, the attenuation in the flux sum of pyruvate leads to an increase in metabolic flux to succinic acid (succinic acid formation rate).

### (1) Recognition of Key Metabolites

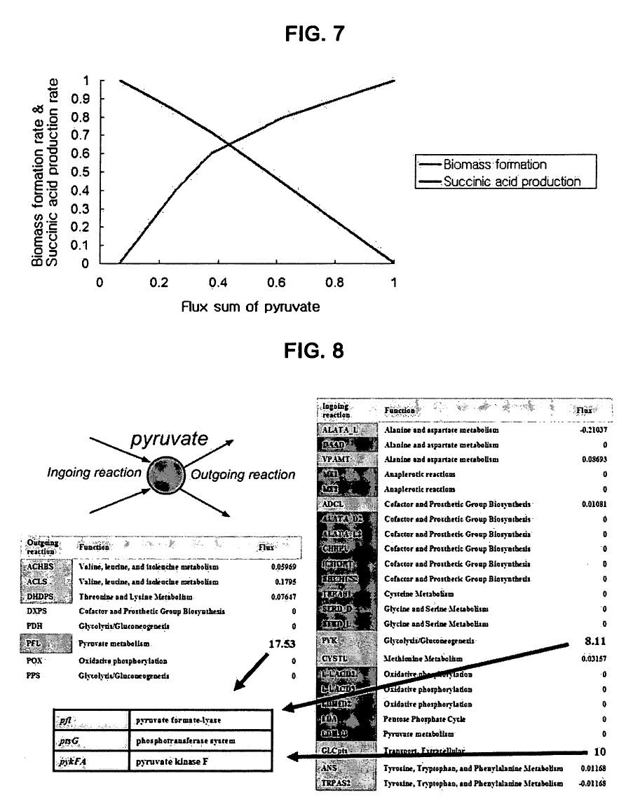

In this Example, key metabolites in the production of succinic acid in anaerobic conditions were examined. It could be found that when all metabolites were perturbed, metabolites showing an increase in the production of succinic acid were pyruvate, acetyle CoA, CoA, formate, ethanol and the like. It can be predicted that as flux sum (φ), which is the metabolite utilization of, in particular, pyruvate, attenuates, the production of succinic acid increases. Accordingly, the profiles of succinic acid production and biomass formation rate when attenuating the flux sum of pyruvate were comparatively examined (see FIG. 7). FIG. 7 shows biomass formation rate of pyruvate and succinic acid formation rate (y-axis) by the perturbation of flux sum (x-axis) of metabolites. From FIG. 7, it could be predicted that as the flux sum of pyruvate attenuates (i.e., metabolic flux producing pyruvate attenuates), the formation of succinic acid increases.

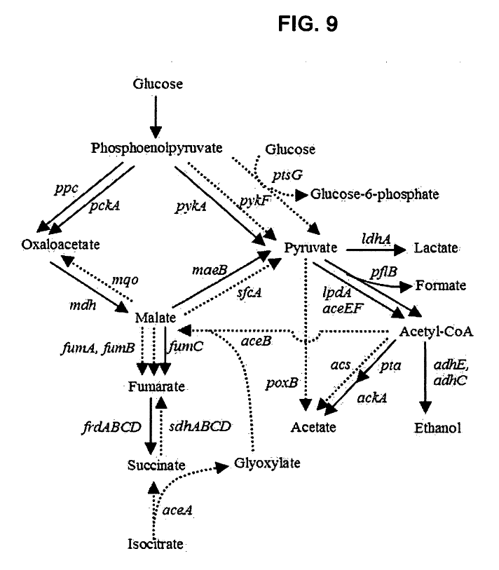

FIG. 8 shows metabolic reactions and the values of metabolic fluxes with respect to pyruvate in E. coli. FIG. 9 shows a metabolic network with respect to pyruvate in E. coli. From FIGS. 8 and 9, metabolic fluxes that highly contribute to flux sum with respect to pyruvate could be found, and it could be predicted from simulation results that the production of succinic acid in E. coli mutant strains having deletions of ptsG, pykF, pykA and pflB genes increases.

Accordingly, to actually construct E. coli mutant strains with deletions of ptsG, pykF, pykA and pflB genes, DNA manipulation standard protocols were used and red recombinase present in the red operon of lambda bacteriophage was used (Sambrook et al., Molecular Cloning: a Laboratory Manual, 3rd edition, 2001; Datsenko et al., Proc. Natl. Acad. Sci. USA, 97:6640, 2000). First, PCR was performed two times using a DNA template containing antibiotic-resistant genes and primers (see Table 1) containing oligonucleotides located upstream and downstream of a target gene to be deleted.

The PCR amplification product was transformed into a parent strain, so that the target gene was replaced with the antibiotic-resistant gene by double homologous recombination, thus constructing a deletion strain having a deletion of the target gene. The constructed strains are shown in Table 2 below. In Table 2, Sp^{r} represents spectinomycin resistance, Tc^{r} represents tetracycline resistance, Cm^{r} represents chloramphenicol resistance, Km^{r} represents kanamycin resistance, and Pm^{r} represents phleomycin resistance.

TABLE 1

| | | | |
|---|---|---|---|
| Template | PCR | Primer | Sequence (5′-3′) |

| | | | |
|---|---|---|---|
| SpR | 1^{st} | SEQ ID NO: 1: | TGC CCG CCG TTG TAT CGC |
| | | PTSG1 | ATG TTA TGG CAG GGG GAT |
| | | | CGA TCC TCT AGA |
| | | SEQ ID NO: 2: | TGC AGC AAC CAG AGC CGG |
| | | PTSG2 | TGC CAT TTC GCT GGG CCG |
| | | | ACA GGC TTT |
| | 2^{nd} | SEQ ID NO: 3: | TGG GCG TCG GTT CCG CGA |
| | | PTSG3 | ATT TCA GCT GGC TGC CCG |
| | | | CCG TTG TAT CGC |
| | | SEQ ID NO: 4: | GAG GTT AGT AAT GTT TTC |
| | | PTSG4 | TTT ACC ACC AAA TGC AGC |
| | | | AAC CAG AGC CGG |
| TcR | 1^{st} | SEQ ID NO: 5: | TGG ACG CTG GCA TGA ACG |
| | | PYKF1 | TTA TGC GTC TGA GGG TAG |
| | | | ATT TCA GTG CAA |
| | | SEQ ID NO: 6: | CGC CTT TGC TCA GTA CCA |
| | | PYKF2 | ACT GAT GAG CCG GGG TTC |
| | | | CAT TCA GGT CGA |
| | 2^{nd} | SEQ ID NO: 7: | CCG AAT CTG AAG AGA TGT |
| | | PYKF3 | TAG CTA AAA TGC TGG ACG |
| | | | CTG GCA TGA ACG |
| | | SEQ ID NO: 8: | AAG TGA TCT CTT TAA CAA |
| | | PYKF4 | GCT GCG GCA CAA CGC CTT |
| | | | TGC TCA GTA CCA |
| CmR | 1^{st} | SEQ ID NO: 9: | GGC ATA CCA TGC CGG ATG |
| | | MQO1 | TGG CGT ATC ATT GGG GTT |
| | | | TAA GGG CAC CAA |
| | | SEQ ID NO: 10: | GAA CTA CGG CGA GAT CAC |
| | | MQO2 | CCG CCA GTT AAT GCC CCG |
| | | | GGC TTT GCG CCG |
| | 2^{nd} | SEQ ID NO: 11: | TGG CGC GTC TTA TCA GCA |
| | | MQO3 | TAC GCC ACA TCC GGC ATA |
| | | | CCA TGC CGG ATG |
| | | SEQ ID NO: 12: | AGC CAC GCG TAC GGA AAT |
| | | MQO4 | TGG TAC CGA TGT GAA CTA |
| | | | CGG CGA GAT CAC |
| KmR | 1^{st} | SEQ ID NO: 13: | CAG TCA GAG AAT TTG ATG |
| | | SDH1 | CAG TTG TGA TTG ATC GGG |
| | | | GGG GGG GGA AAG |
| | | SEQ ID NO: 14: | ATC GGC TCT TTC ACC GGA |
| | | SDH2 | TCG ACG TGA GCG ATC CCA |
| | | | ATT CTG ATT AGA |
| | 2^{nd} | SEQ ID NO: 15: | GTT GTG GTG TGG GGT GTG |
| | | SDH3 | TGA TGA AAT TGC CAG TCA |
| | | | GAG AAT TTG ATG |
| | | SEQ ID NO: 16: | ATC ATG TAG TGA CAG GTT |
| | | SDH4 | GGG ATA ACC GGA ATC GGC |
| | | | TCT TTC ACC GGA |
| PmR | 1^{st} | SEQ ID NO: 17: | GCA CCT TGT GAT GGT GAA |
| | | ACEBA1 | CGC ACC GAA GAA CGA GCT |
| | | | CGG TAC CCG GGC |
| | | SEQ ID NO: 18: | CTT TCG CCT GTT GCA GCG |
| | | ACEBA2 | CCT GAC CGC CAG CAA TAG |
| | | | ACC AGT TGC AAT |
| | 2^{nd} | SEQ ID NO: 19: | GAC GCG CCG ATT ACT GCC |
| | | ACEBA3 | GAT CAG CTG CTG GCA CCT |
| | | | TGT GAT GGT GAA |
| | | SEQ ID NO: 20: | ATC CCG ACA GAT AGA CTG |
| | | ACEBA4 | CTT CAA TAC CCG CTT TCG |
| | | | CCT GTT GCA GCG |
| KmR | 1^{st} | SEQ ID NO: 21: | CAC CTG GTT GTT TCA GTC |
| | | PYKA1 | AAC GGA GTA TTA CAT CGG |
| | | | GGG GGG GGG AAA G |
| | | SEQ ID NO: 22: | GTG GCG TTT TCG CCG CAT |
| | | PYKA2 | CCG GCA ACG TAC ATC CCA |
| | | | ATT CTG ATT AG |
| | 2^{nd} | SEQ ID NO: 23: | TTA TTT CAT TCG GAT TTC |
| | | PYKA3 | ATG TTC AAG CAA CAC CTG |
| | | | GTT GTT TCA GTC |
| | | SEQ ID NO: 24: | GTT GAA CTA TCA TTG AAC |
| | | PYKA4 | TGT AGG CCG GAT GTG GCG |
| | | | TTT TCG CCG CAT C |

TABLE 2

| Strains or plasmids | Explanation |
|---|---|
| E. coli W3110 | Coli Genetic Stock Center strain No. 4474 |
| E. coli W3110GFA | ptsG::Sp^{r}, pykF::Tc^{r}, pykA::Km^{r} |
| E. coli W3110GFAP | ptsG::Sp^{r}, pykF::Tc^{r}, pykA::Km^{r}, pflB::Cm^{r} |

W3110: Coli Genetic Stock Center strain No. 4474
W3110GFA: a mutant of W3110 strain with deletions of ptsG pykF and pykA
W3110GFAP: a mutant of W3110 strain with deletions of ptsG pykF, pykA and pflB

Each of the mutant strains constructed by the above method was cultured at an initial glucose concentration of 60 mM in anaerobic conditions for 24 hours and examined for the concentration of residual glucose and the concentrations of succinic acid, lactate, formate, acetate and ethanol (see Table 3). As a result, as shown in Table 3, it could be found that the ratio of succinic acid relative to other organic acids (S/A ratio) in the mutant strain having deletions of ptsG, pykF and pykA was 9.23 times higher than that in the wild-type strain, and the S/A ratio in the mutant strain having deletions of ptsG pykF, pykA and pflB was 12.60 times higher than that in the wild-type strain.

TABLE 3
Ratio of each organic acid in actual test results

| | | Concentration of fermentation substrate or products | | | | | | | |
|---|---|---|---|---|---|---|---|---|---|
| | | (mM)^{a} | | | | | | Ratio of | |
| | | | Succinic | | | | | succinic | |
| Strains | OD₆₀₀ | Glucose^{b} | acid | Lactate | Formate | Acetate | Ethanol | acid^{c} | Fold^{d} |
| W3110 | 1.79 ± 0.11 | 5.07 ± 0.45 | 2.43 ± 0.03 | 10.62 ± 2.42 | 88.03 ± 0.42 | 40.10 ± 0.20 | 5.77 ± 0.06 | 0.017 | 1 |
| W3110 | 0.99 ± 0.04 | 0.41 ± 0.05 | 17.35 ± 0.03 | 10.67 ± 0.53 | 50.36 ± 3.00 | 30.25 ± 1.47 | 4.14 ± 0.42 | 0.15 | 9.23 |
| GFA | | | | | | | | | |
| W3110 | 0.682 ± 0.16 | 12.2 ± 0.04 | 8.45 ± 0.05 | 21.4 ± 0.88 | — | 4.1 ± 0.04 | 5.5 ± 0.33 | 0.214 | 12.6 |
| GFAP | | | | | | | | | |

^{a}cultured in anaerobic conditions for 24 hours
^{b}measured concentration of residual glucose (initial glucose concentration: 50 mM)
^{c}calculated by an equation of succinic acid/(succinic acid + lactate + formate + acetate + ethanol)
^{d}calculated by an equation of ratio of succinic acid/0.017 (ratio of succinic acid in wild-type strain)

From the above results, it could be found that the production yield of a useful substance can be significantly increased by a method comprising defining the metabolite utilization of a host organism for producing the useful substance as flux sum (φ), perturbing the flux sum to screen key metabolites that increase the production yield of the useful substance, and manipulating the genes of the host organism based on the screened key metabolites so as to construct a mutant organism.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

### INDUSTRIAL APPLICABILITY

As described in detail above, according to the present invention, the changes of specific metabolites can be exactly predicted, so that it is possible to produce a useful substance with high efficiency by introducing and/or amplifying and/or deleting genes expressing enzymes associated with the specific metabolites. In addition, it is also possible to increase the production of a useful substance by adding specific metabolites during culture. Furthermore, according to the present invention, the cause of problems or side effects occurring after introducing and/or deleting genes expressing specific enzymes can be easily found, and on the basis of this, problems that can occur in metabolic manipulation can be predicted and solved prior to actual experiments, so that a strain can be more effectively improved.

1. A method for screening key metabolites involved in an increase in production of a useful substance, the method comprising the steps of:
(a) selecting a non-human host organism for producing a useful target substance, and constructing a metabolic network model of the selected organism;
(b) defining utilization of each metabolite of the constructed metabolic network model as a flux sum (φ) represented by equation 1 below:
$${Φ}_{i} = \vert {f}_{in} \vert \\ = \vert {f}_{out} \vert \\ = 1 / 2 {\sum }_{j}^{ } \vert {S}_{ij} {v}_{j} \vert \qquad [ Equation 1 ]$$
wherein φᵢ represents the utilization of i^{th} metabolite, fᵢₙ represents a metabolic flux of a reaction where a relevant metabolite is consumed with respect to the i^{th} metabolite, fₒᵤₜ represents a metabolic flux of a reaction where the useful target substance is produced with respect to the i^{th} metabolite, Sᵢⱼ represents a stoichiometric coefficient of the i^{th} metabolite in the j^{th} reaction, and vⱼ represents a metabolic flux vector of the j^{th} pathway,
and determining the value φ of the metabolites; and
(c) perturbing the value φ of the metabolites so as to screen key metabolites involved in increasing the production yield of the useful substance.

2. The method for screening key metabolites involved in an increase in the production of a useful substance according to claim 1, wherein the perturbation in the step (c) is performed by increasing and/or attenuating the value φ.

3. The method for screening key metabolites involved in an increase in the production of a useful substance according to claim 2, wherein if an attenuation in the value φ of a specific metabolite leads to an increase in the yield of a useful target product, the specific metabolite is clustered and screened.

4. The method for screening key metabolites involved in an increase in the production of a useful substance according to claim 2, wherein if an increase in the value φ of the specific metabolite leads to an increase in the yield of the useful target product, the specific metabolite is clustered and screened.

5. The method for screening key metabolites involved in an increase in the production of a useful substance according to claim 2, wherein if an increase or attenuation in the value φ of metabolites leads to the yield of the useful target product, the metabolites are clustered and screened.

6. The method for screening key metabolites involved in an increase in the production of a useful substance according to claim 1, wherein the said fᵢₙ and fₒᵤₜ are represented by Equations 2 and 3 below, respectively:
$$\begin{matrix}{f}_{in} = {\sum }_{j}^{ingoing} \vert {S}_{ij} {v}_{j} \vert & [ Equation 2 ] \\ {f}_{out} = {\sum }_{j}^{outgoing} \vert {S}_{ij} {v}_{j} \vert & [ Equation 3 ]\end{matrix}$$
wherein Sᵢⱼ represents the stoichiometric coefficient of the i^{th} metabolite in the j^{th} reaction, and vⱼ represents the metabolic flux vector of j^{th} pathway.

7. The method for screening key metabolites involved in an increase in the production of a useful substance according to claim 1, wherein the host organism is a microorganism.

8. The method for screening key metabolites involved in an increase in the production of a useful substance according to claim 1, wherein the useful substance is succinic acid, and the host organism comprises a succinic acid producing microorganism

9. A method for improving an organism producing a useful substance, the method comprising the steps of:
(a) selecting a host organism for producing a useful target substance, and constructing a metabolic network model of the selected organism;
(b) defining the utilization of each metabolite of the constructed metabolic network model as a flux sum (φ) represented by Equation 1 below:
$${Φ}_{i} = \vert {f}_{in} \vert \\ = \vert {f}_{out} \vert \\ = 1 / 2 {\sum }_{j}^{ } \vert {S}_{ij} {v}_{j} \vert$$
wherein φᵢ represents the utilization of i^{th} metabolite, fᵢₙ represents a metabolic flux of a reaction where a relevant metabolite is consumed with respect to the i^{th} metabolite, fₒᵤₜ represents a metabolic flux of a reaction where the useful target substance is produced with respect to the i^{th} metabolite, Sᵢⱼ represents a stoichiometric coefficient of the i^{th} metabolite in the j^{th} reaction, and vⱼ represents a metabolic flux vector of j^{th} pathway,
and determining the value φ of the metabolites;
(c) clustering and screening a specific metabolite, if an attenuation in the value φ of the specific metabolite leads to an increase in the yield of the useful target substance;
(d) selecting genes to be deleted from a metabolic network associated with the specific metabolite screened in the step (c); and
(e) deleting the genes selected in the step (d) from the host organism so as to construct a mutant of the host organism.

10. The method for improving an organism producing a useful substance according to claim 9, which additionally comprises the step of: (f) culturing the mutant constructed in the step (e) so as to verify the production of the useful substance.

11. The method for improving an organism producing a useful substance according to claim 9, wherein the said fᵢₙ and fₒᵤₜ are represented by Equations 2 and 3 below, respectively:
$$\begin{matrix}{f}_{in} = {\sum }_{j}^{ingoing} \vert {S}_{ij} {v}_{j} \vert & [ Equation 2 ] \\ {f}_{out} = {\sum }_{j}^{outgoing} \vert {S}_{ij} {v}_{j} \vert & [ Equation 3 ]\end{matrix}$$
wherein Sᵢⱼ represents the stoichiometric coefficient of the i^{th} metabolite in the j^{th} reaction, and vⱼ represents the metabolic flux vector of j^{th} pathway.

12. The method for improving an organism producing a useful substance according to claim 9, wherein the host organism comprises a microorganism.

13. The method for improving an organism producing a useful substance according to claim 9, wherein the useful substance is succinic acid, and the host organism is a succinic acid producing microorganism.

14. The method for improving an organism producing a useful substance according to claim 9, comprising deleting at least one gene for the production of succinic acid, wherein the at least one gene to be deleted for the production of succinic acid is selected from the group consisting of ptsG, pflB, pykF and pykA.

15. A method for improving an organism producing a useful substance, the method comprising the steps of:
(a) selecting a host organism for producing a useful target substance, and constructing a metabolic network model of the selected organism;
(b) defining the utilization of each metabolite of the constructed metabolic network model as a flux sum (φ) represented by Equation 1 below:
$${Φ}_{i} = \vert {f}_{in} \vert \\ = \vert {f}_{out} \vert \\ = 1 / 2 {\sum }_{j}^{ } \vert {S}_{ij} {v}_{j} \vert \qquad [ Equation 1 ]$$
wherein φᵢ represents the utilization of i^{th} metabolite, fᵢₙ represents a metabolic flux of a reaction where a relevant metabolite is consumed with respect to the i^{th} metabolite, fₒᵤₜ represents a metabolic flux of a reaction where the useful target substance is produced with respect to the i^{th} metabolite, Sᵢⱼ represents a stoichiometric coefficient of the i^{th} metabolite in the j^{th} reaction, and vⱼ represents a metabolic flux vector of j^{th} pathway, and determining the value φ of the metabolites;
(c) clustering and screening a specific metabolite, if an increase in the value φ of the specific metabolite leads to an increase in the yield of the useful target substance;
(d) selecting genes to be amplified from a metabolic network associated with the specific metabolite screened in the step (c); and
(e) introducing the genes selected in the step (d) into the host organism and/or amplifying the genes in the host organism, so as to construct a mutant of the host organism.

16. The method for improving an organism producing a useful substance according to claim 15, which additionally comprises the step of: (f) culturing the mutant constructed in the step (e) so as to verify the production of the useful substance.

17. The method for improving an organism producing a useful substance according to claim 15, wherein the said fᵢₙ and fₒᵤₜ are represented by Equations 2 and 3 below, respectively:
$$\begin{matrix}{f}_{in} = {\sum }_{j}^{ingoing} \vert {S}_{ij} {v}_{j} \vert & [ Equation 2 ] \\ {f}_{out} = {\sum }_{j}^{outgoing} \vert {S}_{ij} {v}_{j} \vert & [ Equation 3 ]\end{matrix}$$
wherein Sᵢⱼ represents the stoichiometric coefficient of the i^{th} metabolite in the j^{th} reaction, and vⱼ represents the metabolic flux vector of j^{th} pathway.

18. The method for improving an organism producing a useful substance according to claim 15, wherein the host organism comprises a microorganism.

19. The method for improving an organism producing a useful substance according to claim 15, wherein the useful substance is succinic acid, and the host organism is a succinic acid producing microorganism.

20. A method for improving an organism producing a useful substance, the method comprising the steps of:
(a) selecting a host organism for producing a useful target substance, and constructing a metabolic network model of the selected organism;
(b) defining the utilization of each metabolite of the constructed metabolic network as a flux sum φ represented by Equation 1 below:
$${Φ}_{i} = \vert {f}_{in} \vert \\ = \vert {f}_{out} \vert \\ = 1 / 2 {\sum }_{j}^{ } \vert {S}_{ij} {v}_{j} \vert \qquad [ Equation 1 ]$$
wherein φᵢ represents the utilization of i^{th} metabolite, fᵢₙ represents a metabolic flux of a reaction where a relevant metabolite is consumed with respect to the i^{th} metabolite, fₒᵤₜ represents a metabolic flux of a reaction where the useful target substance is produced with respect to the i^{th} metabolite, Sᵢⱼ represents a stoichiometric coefficient of the i^{th} metabolite in the j^{th} reaction, and vⱼ represents a metabolic flux vector of j^{th} pathway and determining the value φ of the metabolites;
(c) clustering and screening metabolites, if an attenuation and increase in the value φ of the metabolites leads to an increase in the yield of the useful target substance;
(d) selecting genes to be deleted and genes to be amplified from a metabolic network associated with the metabolites screened in the step (c); and
(e) deleting the genes to be deleted, which are selected in the step (d) from the host organism while introducing the genes to be amplified into the host organism and/or amplifying the genes in the host organism, so as to construct a mutant of the host organism.

21. The method for improving an organism producing a useful substance according to claim 20, which additionally comprises the step of: (f) culturing the mutant constructed in the step (e) so as to verify the production of the useful substance.

22. The method for improving an organism producing a useful substance according to claim 20, wherein the said fᵢₙ and fₒᵤₜ are represented by Equations 2 and 3 below, respectively:
$$\begin{matrix}{f}_{in} = {\sum }_{j}^{ingoing} \vert {S}_{ij} {v}_{j} \vert & [ Equation 2 ] \\ {f}_{out} = {\sum }_{j}^{outgoing} \vert {S}_{ij} {v}_{j} \vert & [ Equation 3 ]\end{matrix}$$
wherein Sᵢⱼ represents the stoichiometric coefficient of the i^{th} metabolite in the j^{th} reaction, and vⱼ represents the metabolic flux vector of j^{th} pathway.

23. The method for improving an organism producing a useful substance according to claim 20, wherein the host organism comprises a microorganism.

24. The method for improving an organism producing a useful substance according to claim 20, wherein the useful substance is succinic acid, and the host organism is a succinic acid producing microorganism.

25. A method for producing a useful substance, wherein the method comprises culturing the organism improved by the method of claim 9.

26. A method for producing a useful substance by culture of an organism, which comprises supplying a metabolite in a culture process, and screening the metabolite, by a process comprising the steps of:
(a) selecting a non-human host organism for producing a useful target substance, and constructing a metabolic network model of the selected organism;
(b) defining the utilization of each metabolite of the constructed metabolic network model as a flux sum (φ) represented by Equation 1 below:
$${Φ}_{i} = \vert {f}_{in} \vert \\ = \vert {f}_{out} \vert \\ = 1 / 2 {\sum }_{j}^{ } \vert {S}_{ij} {v}_{j} \vert \qquad [ Equation 1 ]$$
wherein φᵢ represents the utilization of i^{th} metabolite, fᵢₙ represents a metabolic flux of a reaction where a relevant metabolite is consumed with respect to the i^{th} metabolite, fₒᵤₜ represents a metabolic flux of a reaction where the useful target substance is produced with respect to the i^{th} metabolite, Sᵢⱼ represents a stoichiometric coefficient of the i^{th} metabolite in the j^{th} reaction, and vⱼ represents a metabolic flux vector of j^{th} pathway,
and determining the value φ of the metabolites; and
(c) clustering and screening a specific metabolite, if an increase in the value φ of the specific metabolite leads to the yield of the useful target substance.

27. The method for producing a useful substance by the culture of an organism, which comprises supplying the metabolite in the culture process, according to claim 26, wherein the said fᵢₙ and fₒᵤₜ are represented by Equations 2 and 3 below, respectively:
$$\begin{matrix}{f}_{in} = {\sum }_{j}^{ingoing} \vert {S}_{ij} {v}_{j} \vert & [ Equation 2 ] \\ {f}_{out} = {\sum }_{j}^{outgoing} \vert {S}_{ij} {v}_{j} \vert & [ Equation 3 ]\end{matrix}$$
wherein Sᵢⱼ represents the stoichiometric coefficient of the i^{th} metabolite in the j^{th} reaction, and vⱼ represents the metabolic flux vector of j^{th} pathway.
